(19) United States
(12) Patent Application Publication
(10) Pub. No.: US 2024/0019428 A1
(43) Pub. Date: Jan. 18, 2024

(54) CHIP FOR QUANTITATIVE DETECTION OF NEUTRALIZING ANTIBODY AND MANUFACTURING METHOD THEREOF

(71) Applicant: NATIONAL APPLIED RESEARCH LABORATORIES, TAIPEI CITY (TW)

(72) Inventors: HUEIH-MIN CHEN, HSINCHU (TW); CHIH-CHAO YANG, HSINCHU (TW); CHIH-YU HUANG, HSINCHU (TW)

(21) Appl. No.: 18/052,321

(22) Filed: Nov. 3, 2022

(30) Foreign Application Priority Data
Jul. 18, 2022 (TW) 111126868

(51) Int. Cl. G01N 33/543

(52) U.S. Cl. CPC G01N 33/54393; G01N 33/5438; G01N 33/5436; G01N 2610/00; G01N 2333/165

(57) ABSTRACT

The present invention provides a detection chip for quantitative detection of neutralizing antibody and manufacturing method thereof. A sensing layer is disposed on a circuit layer. A shielding layer corresponds a shielding part on the sensing layer to form a sensing area. The surface of the sensing area is hydroxylated to form a self-assembled monolayer film including the aldehyde group. A protein solution is dripped on the sensing area. An external electric field is applied to the sensing layer at an external angle with respect to the normal of the substrate to deflect protein molecules in the protein solution correspondingly. This structure can be applied to rapid and quantitative detection. According to various embodiments of the present invention, the sensing efficiency of the detection chip can be enhanced.

### FIELD OF THE INVENTION

The present invention relates generally to a chip for quantitative detection of neutralizing antibody and the manufacturing method thereof, and particularly to a chip for quantitative detection of target object and the manufacturing method thereof.

### BACKGROUND OF THE INVENTION

The sensing performance of detection chips is mainly determined by the stability, uniformity, and orientation of protein molecule bound thereon. The self-assembled monolayer (SAM) technology is capable of fixing monolayer protein molecules on chip surface using covalent bonds with stability and uniformity. To further improve the performance of protein chips, the orientation of protein molecules should be addressed.

Proteins are stereoscopic biological molecules. Their conformation has much to do with their functions. For example, the binding of protein molecules must be achieved via special binding sites in their structure. Thereby, the binding of protein molecules is directional. When protein molecules are used to manufacture detection chips, if the binding sites of protein molecules are hidden at the bottom instead of exposing to the chip surface, the binding efficiency between protein molecules and the target ligands will be reduced.

Unfortunately, while fixing protein molecules on detection chips, if no external force is introduced, protein molecules will be distributed on detection chips with random orientations and hence lowering the binding efficiency between the protein molecules on detection chips and the target ligands to be detected and deteriorating the sensing efficiency of detection chips. Accordingly, it is urged by the industry to develop a structure design capable of improving the binding efficiency between protein molecules on chips and target ligands.

To solve the above problem according to the prior art, the present invention provides a chip for quantitative detection of neutralizing antibody and the manufacturing method thereof. A sensing layer is disposed on a circuit layer on a substrate. A shielding layer is then disposed on the sensing layer correspondingly or between the circuit layer and the substrate correspondingly. The shielding layer shields a part of the sensing layer to form a sensing area. By applying an external electric field at an external angle, protein molecules are moved and deflected according to their charge characteristics to become unidirectional. They are then fixed in the sensing area and thus achieving rapid and quantitative detection.

### SUMMARY

An objective of the present invention is to provide a method for manufacturing a chip for quantitative detection of neutralizing antibody. The spike protein solution of coronaviruses is dripped on the sensing area of the detection chip. By applying an external electric filed at an external angle, the protein molecules in the protein solution are moved and deflected to become unidirectional and bind with a self-assembled monolayer film. The external angle deflects the protein molecules in the protein solution to the optimum exposed direction of bindable receptor binding domain. This method can manufacture rapid and quantitative detection chips.

An objective of the present invention is to provide a chip for quantitative detection of neutralizing antibody. A sensing layer is disposed on a circuit layer on a substrate. A shielding layer is then disposed on the sensing layer correspondingly or between the circuit layer and the substrate correspondingly. The shielding layer shields a part of the sensing layer to form a sensing area. The shielding layer is used to limit the area for protein fixation and thus achieving rapid and quantitative detection.

An objective of the present invention is to provide a chip for quantitative detection of neutralizing antibody. A sensing layer is disposed on a circuit layer on a substrate. A shielding layer is then disposed between the circuit layer and the substrate correspondingly. The shielding layer shields the substrate and limit the sensing layer to form a sensing area for sensing the area of target ligands under detection. Thereby, the sensing efficiency of detection chips can be improved.

To achieve the above objectives and efficacies, the present invention provides a method for manufacturing a chip for quantitative detection of neutralizing antibody applied to binding protein molecules in a protein solution. The manufacturing method comprises steps of: dripping the protein solution to a chip for quantitative detection of neutralizing antibody; and applying an external electric field to the chip for quantitative detection of neutralizing antibody at an external angle with respect to the normal of the chip for quantitative detection of neutralizing antibody and the external electric field deflecting the protein molecules corresponding to the external angle. The method can be used to manufacture a chip for quantitative detection of neutralizing antibody.

To achieve the above objectives and efficacies, the present invention provides a chip for quantitative detection of neutralizing antibody, which comprises a substrate, a plurality of circuit layers, a sensing layer, and a shielding layer. The circuit layers are disposed on the substrate. The sensing layer is disposed on the substrate and on the plurality of circuit layers. The shielding layer is disposed on the sensing layer and includes an opening corresponding to the plurality of circuit layers. The opening forms a sensing area on the sensing layer. By using the structure, the sensing efficiency of the detection chip can be improved and the content of a target object can be detected quantitatively.

To achieve the above objectives and efficacies, the present invention provides a chip for quantitative detection of neutralizing antibody, which comprises a substrate, a plurality of circuit layers, a plurality of sensing layers, and a shielding layer. The plurality of circuit layers are disposed on the substrate. The plurality of sensing layers are disposed on the plurality of circuit layers, respectively. The shielding layer is disposed on the substrate, between the plurality of circuit layers, respectively, and on the plurality of sensing layers. The shielding layer includes an opening corresponding to the plurality of circuit layers, respectively. The opening forms a sensing area on the plurality of sensing layers, respectively. By using the structure, the sensing efficiency of the detection chip can be improved and the content of a target object can be detected quantitatively.

To achieve the above objectives and efficacies, the present invention provides a chip for quantitative detection of neutralizing antibody, which comprises a substrate, a plurality of circuit layers, a plurality of sensing layers, and a shielding layer. The plurality of circuit layers are disposed on the substrate. The plurality of sensing layers cover the outer side of the plurality of circuit layers, respectively. The shielding layer is disposed on the substrate and between the plurality of circuit layers to form a plurality of sensing areas on the plurality of sensing layers. By using the structure, the sensing efficiency of the detection chip can be improved and the content of a target object can be detected quantitatively.

To achieve the above objectives and efficacies, the present invention provides a chip for quantitative detection of neutralizing antibody, which comprises a substrate, a shielding layer, a plurality of circuit layers, and a plurality of sensing layers. The shielding layer is disposed on the substrate. The plurality of circuit layers are disposed on the shielding layer. The plurality of sensing layers cover the outer side of the plurality of circuit layers, respectively, to form a plurality of sensing areas on the plurality of sensing layers. By using the structure, the sensing efficiency of the detection chip can be improved and the content of a target object can be detected quantitatively.

According to an embodiment of the present invention, before the step of dripping the protein solution to a chip for quantitative detection of neutralizing antibody, which step is used to bind protein molecules to a sensing area of the chip for quantitative detection of neutralizing antibody, the present invention further comprises steps of hydroxylating the sensing area of the chip for quantitative detection of neutralizing antibody; and forming a self-assembled monolayer film including the aldehyde group for binding the protein molecules to the sensing area.

According to an embodiment of the present invention, after hydroxylating the surface of the sensing area and forming a self-assembled monolayer film including the aldehyde group, drip the protein solution to the sensing area and apply an external electric field to the sensing layer at an external angle with respect to the normal of the substrate for deflecting the protein molecules in the protein solution to the optimum exposed direction of receptor binding domain bindable with target ligands and binding with the self-assembled monolayer film.

According to an embodiment of the present invention, after hydroxylating the surfaces of the plurality of sensing areas and forming a self-assembled monolayer film including the aldehyde group, drip the protein solution to the sensing area and apply an external electric field to the plurality of sensing areas at an external angle with respect to the normal of the substrate for deflecting the protein molecules in the protein solution and binding with the self-assembled monolayer film.

According to an embodiment of the present invention, after hydroxylating the surfaces of the plurality of sensing areas and forming the self-assembled monolayer film, the present invention further forming a cross-linked molecular film on the self-assembled monolayer film.

According to an embodiment of the present invention, the material of the shielding layer is selected from the group consisting of silicon mononitride and silicon oxynitride.

According to an embodiment of the present invention, the receptor binding domain of the protein molecules belongs to the superfamily and subfamily of CoV_Spike_S1_RBD.

According to an embodiment of the present invention, the external angle with respect to the normal of the chip for quantitative detection of neutralizing antibody is between −15° and 15°.

### BRIEF DESCRIPTION OF THE DRAWINGS

FIG. 1 shows a schematic diagram of the structure according to the first embodiment of the present invention;

FIGS. 2a to 2d show schematic diagrams of protein fixation according to the first embodiment of the present invention;

FIG. 3 shows a schematic diagram of the structure according to the second embodiment of the present invention;

FIGS. 4a to 4d show schematic diagrams of protein fixation according to the second embodiment of the present invention;

FIG. 5 shows a schematic diagram of the structure according to the third embodiment of the present invention;

FIGS. 6a to 6d show schematic diagrams of protein fixation according to the third embodiment of the present invention;

FIG. 7 shows a schematic diagram of the structure according to the fourth embodiment of the present invention;

FIGS. 8a to 8d show schematic diagrams of protein fixation according to the fourth embodiment of the present invention;

FIG. 9 shows a schematic diagram of external electric field according to an embodiment of the present invention;

FIG. 10 shows a flowchart according to an embodiment of the present invention;

FIG. 11 shows another flowchart according to an embodiment of the present invention;

FIG. 12 shows a measurement chart of binding force versus protein angle according to an embodiment of the present invention;

FIGS. 13a to 13b show a measurement chart of quantitative detection of target ligands according to an embodiment of the present invention; and

FIGS. 14a to 14b show schematic diagrams of protein fixation according to another embodiment of the present invention.

### DETAILED DESCRIPTION

In order to make the structure and characteristics as well as the effectiveness of the present invention to be further understood and recognized, the detailed description of the present invention is provided as follows along with embodiments and accompanying figures.

To solve the technical problems according to the prior art as described above, according to the present invention, a shielding layer is disposed on a sensing layer for shielding a part of the sensing layer correspondingly. The shielding layer includes an opening corresponding to the circuit layer. A sensing area is formed on the sensing layer corresponding to the opening. The sensing area is used to bind protein molecules on the sensing layer for detecting the target ligands. According to the present invention, the shielding layer is further disposed between a plurality of circuit layers and a substrate for partitioning the sensing areas on the sensing layer for detecting the target ligands.

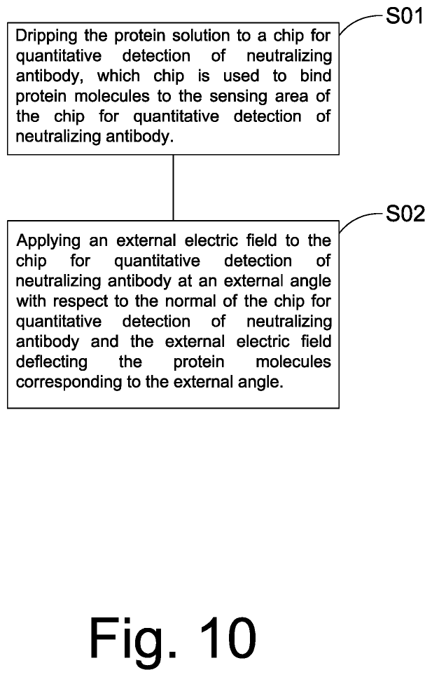

Please refer to FIG. 10, which shows a flowchart according to an embodiment of the present invention. As shown in the figure, the present embodiment relates to a method for manufacturing a chip for quantitative detection of neutralizing antibody. First, a protein solution containing protein molecules should be provided for the manufacturing method. The method for manufacturing a chip for quantitative detection of neutralizing antibody comprises steps of:
Step S01: Dripping the protein solution to a chip for quantitative detection of neutralizing antibody, which chip is used to bind protein molecules to the sensing area of the chip for quantitative detection of neutralizing antibody; and
Step S02: Applying an external electric field to the chip for quantitative detection of neutralizing antibody at an external angle with respect to the normal of the chip for quantitative detection of neutralizing antibody and the external electric field deflecting the protein molecules corresponding to the external angle.

According to the present embodiment, the protein molecules in the protein solution further include spike proteins of coronaviruses with receptor binding domain belonging to the superfamily and subfamily of CoV_Spike_S1_RBD and the National Center for Biotechnology Information (NCBI) registration number c140478.

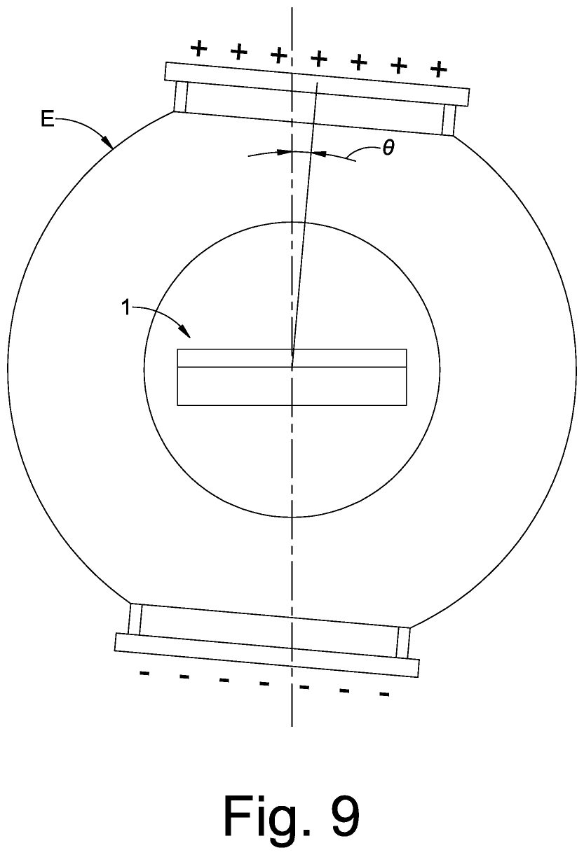

Please refer to FIG. 10 again and to FIG. 9, which shows a schematic diagram of external electric field according to an embodiment of the present invention. As shown in the figures, according to the present embodiment, in the step S01, the protein solution is dripped on a detection chip 1. In the step S02, after the protein solution is dripped on the detection chip 1, apply an external electric field E at an external angle θ with respect to the normal of the detection chip 1 so that the protein molecules in the protein solution can be deflected to the optimum exposed direction of receptor binding domain bindable with target ligands.

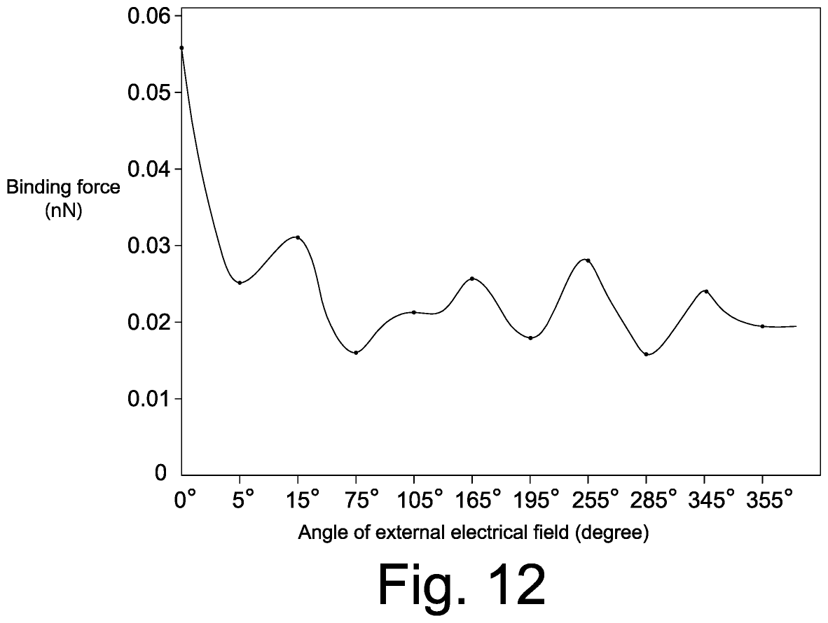

Please refer to FIG. 10 and FIG. 9 again and to FIG. 12, which shows a measurement chart of binding force versus protein angle according to an embodiment of the present invention. As shown in the figures, according to the present embodiment, the external angle θ of the external electric field E is used to deflect protein molecules to the optimum exposed direction of receptor binding domain for fixing to the detection chip 1. As shown in FIG. 12, when the external angle θ of the external electric field E is 0°, the binding force (in units of, for example, nanonewton, nN) between the protein molecules and the detection chip 1 is maximum. Thereby, according to the present embodiment, to have larger binding force, the external angle θ should be in the range between 0° and 15° (clockwise) and 345° and 0° (counterclockwise). In other words, the external angle is between −15° and 15° (clockwise). According to an embodiment, the external angle θ is preferably 0°.

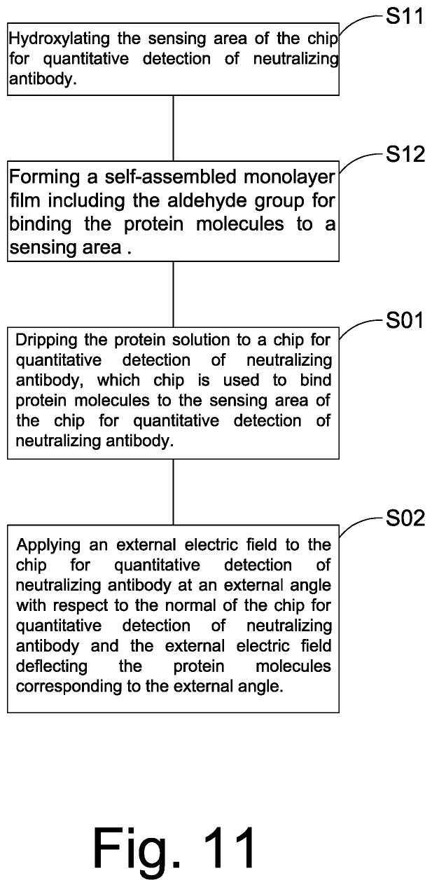

Please refer to FIG. 11, which shows another flowchart according to an embodiment of the present invention. As shown in the figure, according to the present embodiment, before the step S01 of dripping the protein solution to a chip for quantitative detection of neutralizing antibody, the present invention further comprises steps of:
Step S11: Hydroxylating the sensing area of the chip for quantitative detection of neutralizing antibody; and
Step S12: Forming a self-assembled monolayer film including the aldehyde group for binding the protein molecules to a sensing area.

In the step S11, the detection chip 1 includes a sensing area. In addition, before dripping the protein solution, the sensing area of the detection chip 1 is hydroxylated. In the step S12, a self-assembled monolayer film including the aldehyde group is formed on the sensing area of the detection chip 1. Afterwards, the steps S01 and S02 are executed to form covalent bonds between the protein molecules in the protein solution and the self-assembled monolayer film. Then the external electric filed E is used to align the protein molecules to the same external angle θ before fixing the protein molecules to the self-assembled monolayer film in the optimum exposed direction of receptor binding domain.

According to the present embodiment, oxygen plasma is used to process the chip for quantitative detection of neutralizing antibody and hydroxylate the surface thereof. Alternatively, the piranha solution can be used to immerse the chip for quantitative detection of neutralizing antibody and hydroxylate the surface thereof.

According to the present embodiment, triethoxysilylundecanal (TESUA) is used to form a self-assembled monolayer film including the aldehyde group on the chip for quantitative detection of neutralizing antibody.

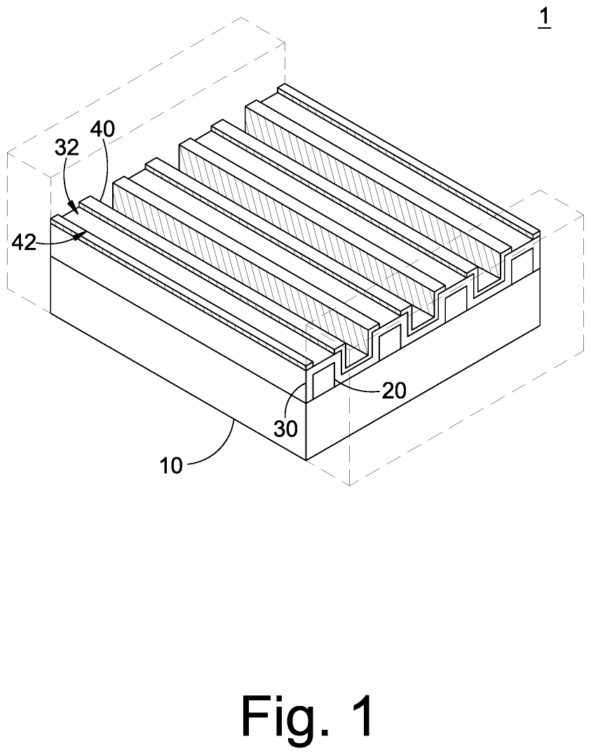

Please refer to FIG. 1, which shows a schematic diagram of the structure according to the first embodiment of the present invention. As shown in the figure, a detection chip 1 corresponding to the manufacturing method according to the first embodiment comprises a substrate 10, a plurality of circuit layers 20, a sensing layer 30, and a shielding layer 40. According to the present embodiment, the material of the substrate 10 includes, but not limited to, silicon.

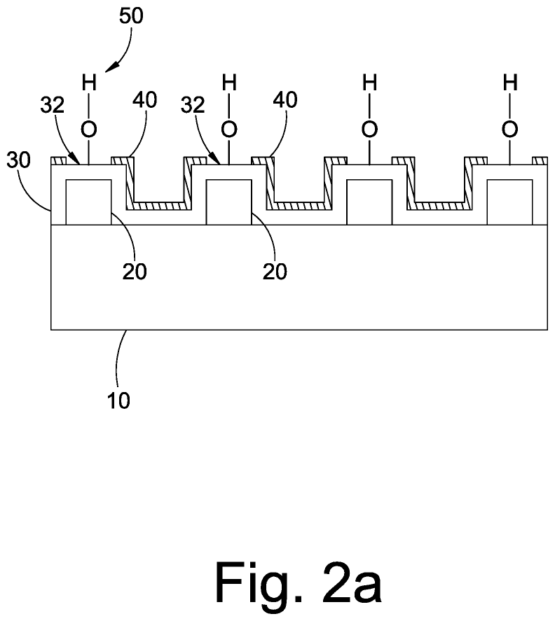

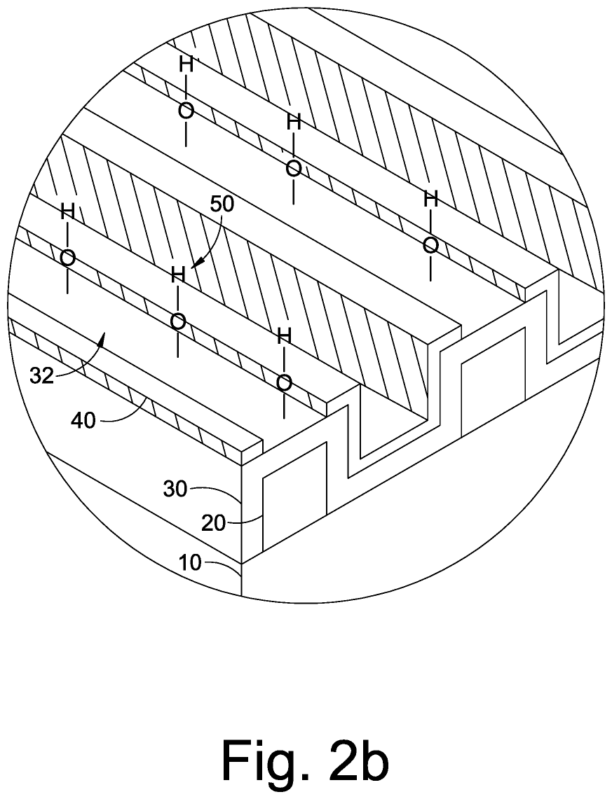

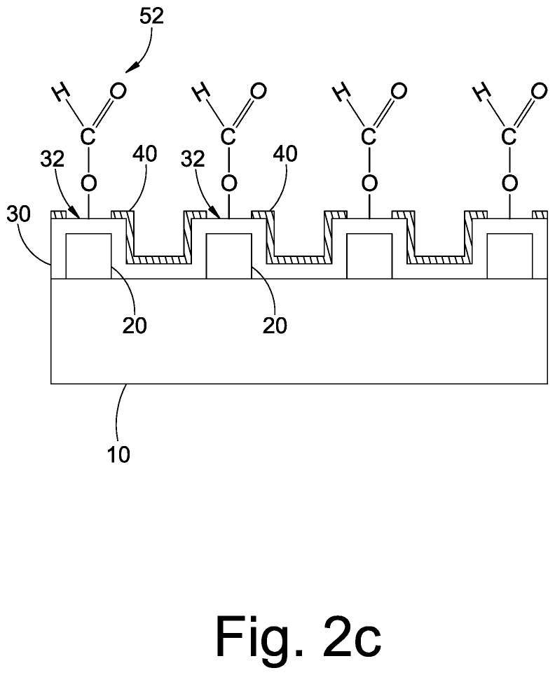

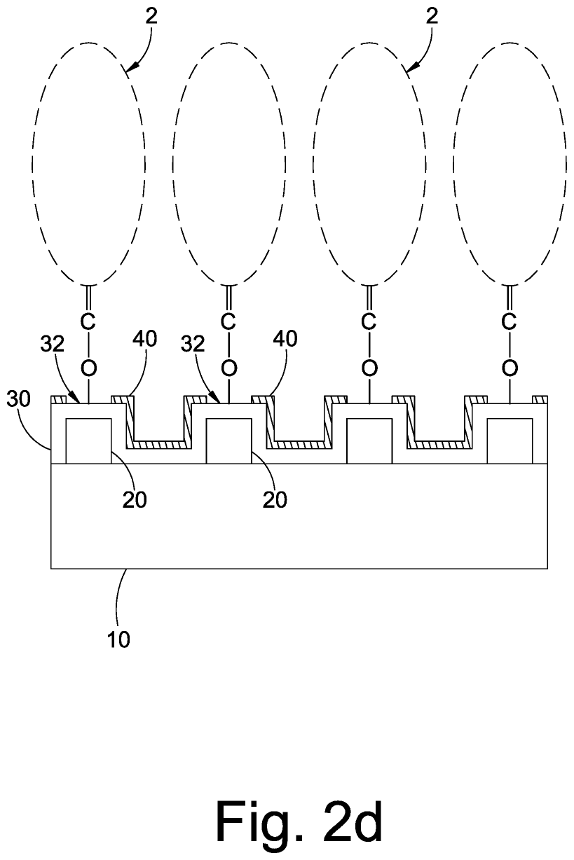

Please refer to FIG. 1 again and to FIGS. 2a to 2d, which show schematic diagrams of protein fixation according to the first embodiment of the present invention. As shown in the figures, according to the present embodiment, the plurality of circuit layers 20 are disposed on the substrate 10. The sensing layer 30 is then disposed on the substrate 10 and on the plurality of circuit layers 20. Namely, the sensing layer 30 covers the surfaces of the substrate 10 and the plurality of circuit layers 20. The shielding layer 40 is disposed on the sensing layer 30. The shielding layer 40 further includes an opening 42 corresponding to the plurality of circuit layers 20. The opening 42 forms a sensing area 32 on the sensing layer 30. According to the present embodiment, the shielding layer 40 on the plurality of circuit layers 20 includes the opening 42, respectively, and hence forming a plurality of openings 42, which correspond to the sensing layer 30 and forming a plurality of sensing areas 32.

Please refer again to FIGS. 2a to 2d and FIG. 9. As shown in the figures, according to the present embodiment, the sensing layer 30 is further connected to a hydroxyl group 50 for hydroxylating the surface thereof. According to the present embodiment, due to the shielding of the shielding layer 40, the hydroxyl group 50 is only disposed on the sensing area 32 of the sensing layer 30 to hydroxylate the surface of the sensing layer 30 in the sensing area 32. Then, a self-assembled monolayer film 52 including the aldehyde group is formed on the hydroxylated sensing area 32. Afterwards, according to the method for manufacturing a chip for quantitative detection of neutralizing antibody as described above, drip the protein solution on the sensing area 32 and apply an external electric field E at an external angle θ to the sensing layer 32 to deflect the protein molecules 2 in the protein solution and bind with the self-assembled monolayer film 52. According to the present embodiment, the external angle θ is the angle between the positive and negative electrodes of the external electric field E and the normal of the substrate 10.

According to the present embodiment, according to the present embodiment, the protein molecules 2 then bind with the target ligands to be detected. By limiting the locations of the hydroxyl group 50 and the protein molecules 2, the binding efficiency between the protein molecules and the target ligands to be detected can be enhanced and thus achieving the efficacy of quantitative detecting target ligands.

According to the present embodiment, according to the present embodiment, as shown in FIG. 12, when the external angle θ of the external electric field E is between −15° and 15°, the binding force of the protein molecules 2 is optimum. Thereby, according to the present embodiment, the external angle θ is between −15° and 15°. According to an embodiment, the external angle θ is preferably 0°.

According to the present embodiment, according to the present embodiment, a plurality of hydroxyl groups 50 and a plurality of protein molecules 2 are disposed in the sensing areas 32 of the sensing layer 30. Nonetheless, the present invention is not limited to the present embodiment.

According to the present embodiment, according to the present embodiment, the material of the plurality of sensing layers 30 is selected from the group consisting of SiO₂, NdAlO₃, Al₂O₃, PrAlO₃, HfO₂, SmAlO₃, BeAl₂O₄, SrTiO₃, (Ba,Sr)TiO₃, Ta₂O₅, CeO₂, TiO₂, La₂O₃, Y₂O₃, LaAlO₃, ZrO₂, and LaScO₃. Nonetheless, the present invention is not limited to the present embodiment.

According to the present embodiment, according to the present embodiment, the material of the shielding layer 40 is selected from the group consisting of SiNx, TiN, CxFy, TaN, SiC, AlN, CHx (hydrogen carbides), α-Si, α-Ge, α-Carbon, BN, and CNx (nitrogen carbides). Nonetheless, the present invention is not limited to the present embodiment. These materials can avoid the hydroxyl group 50 from being disposed on the surface of the shielding layer 40, which will further cause the chip 1 for quantitative detection of neutralizing antibody to generate false signals.

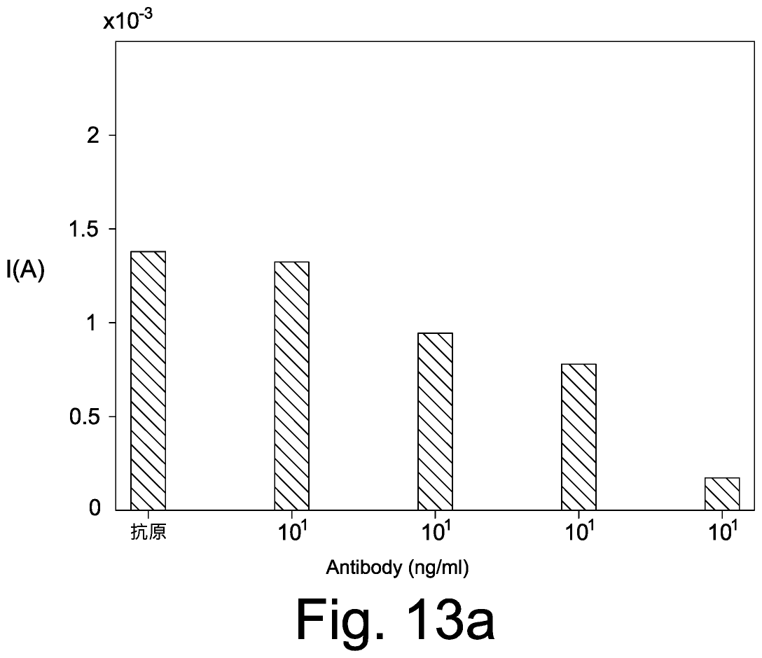

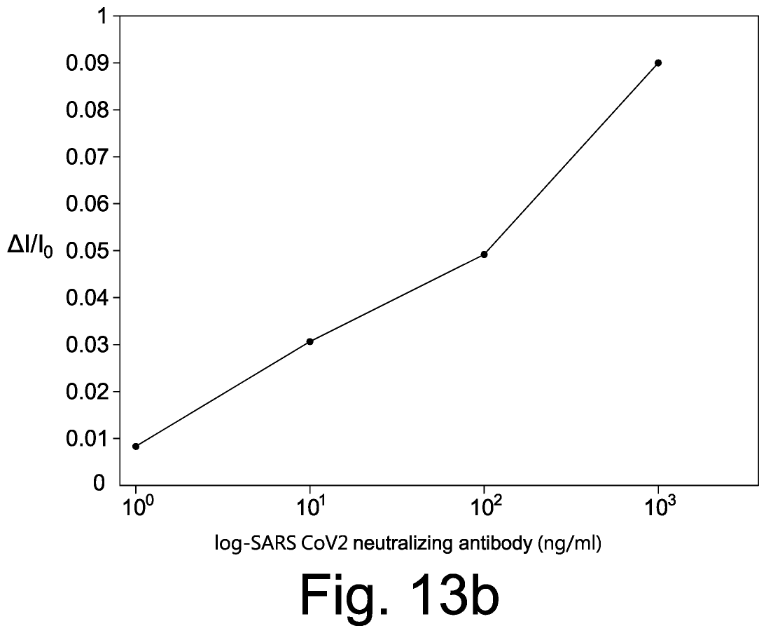

Please refer to FIGS. 13a to 13b, which show a measurement chart of quantitative detection of target ligands according to an embodiment of the present invention. As shown in the figures, the chip for quantitative detection of neutralizing antibody according to the present embodiment is used for quantitative detection of unknown antibody induced by the COVID-19 virus. As shown in FIG. 13a, the present embodiment tests the antibody anti-RBD (MW:˜200 kD) with various concentrations (1 to 1000 ng/ml). The concentration of the antigen is fixed at 1.5 μg/ml (MW: 61.2 kD). As shown in FIG. 13b, after detection by the chip for quantitative detection of neutralizing antibody, the standard curve for different antibodies with known concentrations can be given. Thereby, the concentration of the antibody acquired from human blood or saliva can be confirmed by interpolation of the standard curve and thus achieving the purpose of quantitative detection of the concentration of the target ligands (for example, the unknown antibody induced by the COVID-19 virus or vaccine).

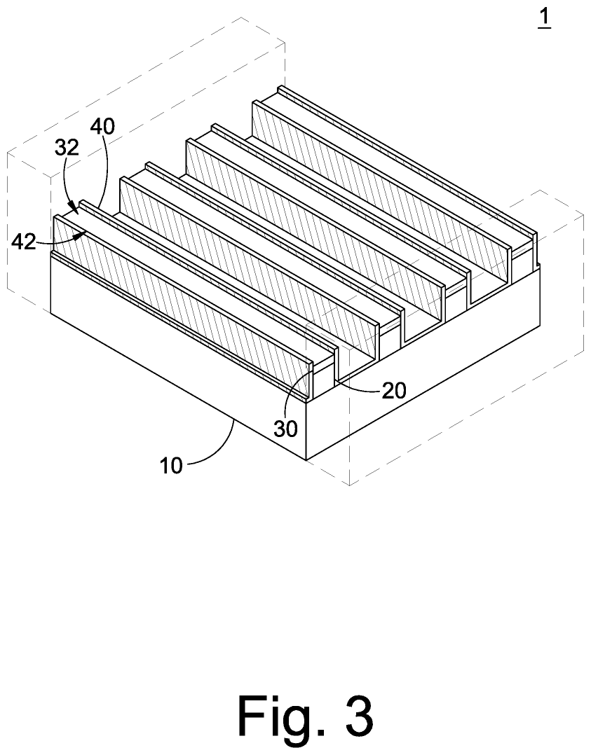

Please refer to FIG. 3, which shows a schematic diagram of the structure according to the second embodiment of the present invention. As shown in the figure, a detection chip 1 corresponding to the manufacturing method according to the second embodiment comprises a substrate 10, a plurality of circuit layers 20, a plurality of sensing layers 30, and a shielding layer 40. According to the present embodiment, the material of the substrate 10 includes, but not limited to, silicon.

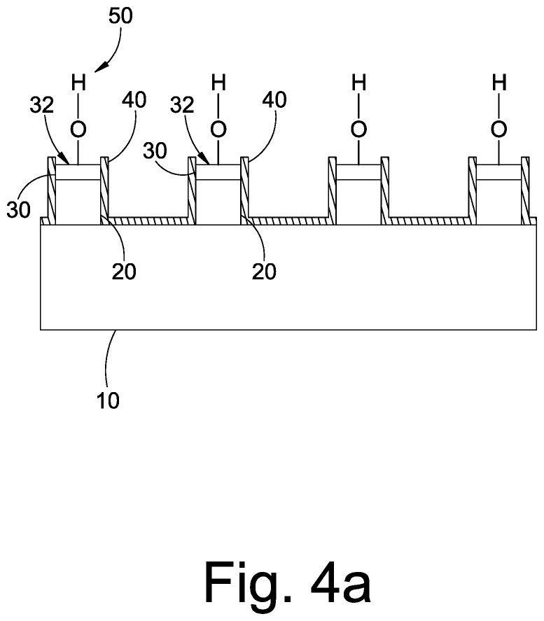

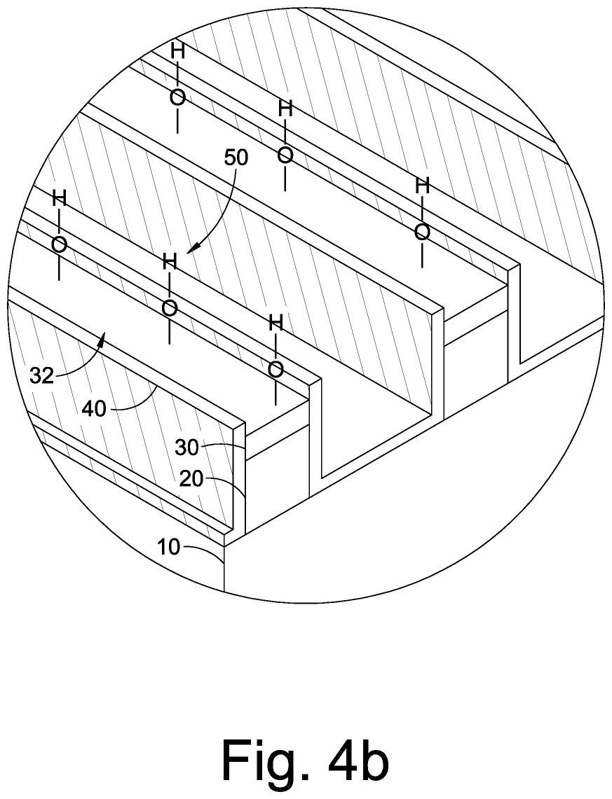

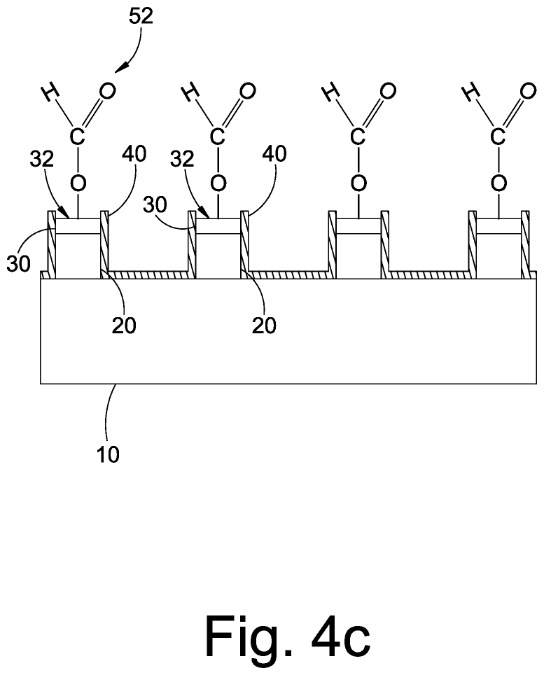

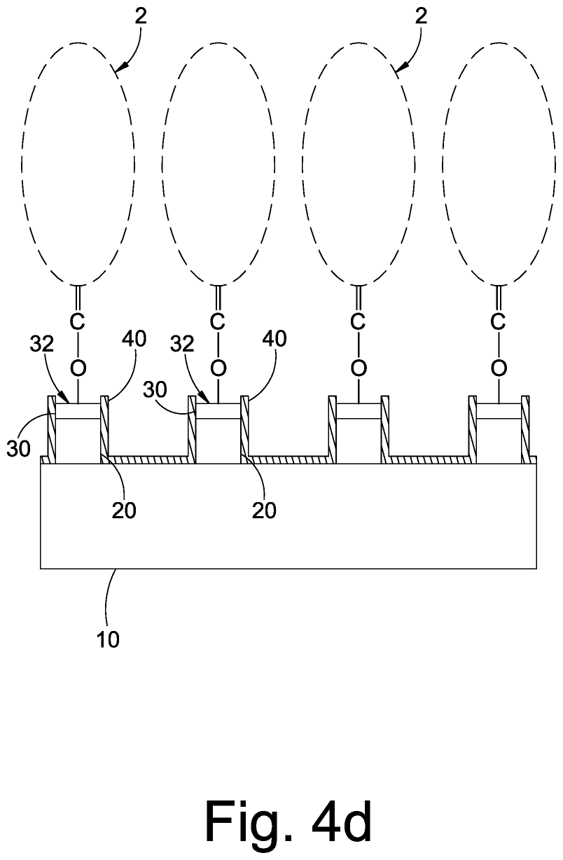

Please refer to FIG. 3 again and to FIGS. 4a to 4d, which show schematic diagrams of protein fixation according to the second embodiment of the present invention. As shown in the figures, according to the present embodiment, the plurality of circuit layers 20 are disposed on the substrate 10. The plurality of sensing layers 30 are disposed on the plurality of circuit layers 20, respectively. Namely, one of the plurality of sensing layers 30 covers the surface of one of the plurality of circuit layers 20. The shielding layer 40 is disposed on the substrate 10, between the plurality of circuit layers 20, and on the sensing layer 30. The shielding layer 40 includes an opening 42 corresponding to the plurality of circuit layers 20, respectively. The opening 42 forms a sensing area 32 on the plurality of sensing layers 30, respectively. According to the present embodiment, the shielding layer 40 on the plurality of circuit layers 20 includes the opening 42, respectively, and hence forming a plurality of openings 42, which correspond to the plurality of sensing layers 30 and forming a plurality of sensing areas 32.

The difference between the present embodiment and the first embodiment as described above is that, according to the present embodiment, the plurality of sensing layers 30 are disposed on the corresponding circuit layers 20 only, and the shielding layer 40 covers the substrate 10 and the side surfaces between the plurality of circuit layers 20 for further shrinking the size of the chip 1 for quantitative detection of neutralizing antibody and reducing waste of materials.

Please refer again to FIGS. 4a to 4d and FIG. 9. As shown in the figures, according to the present embodiment, the plurality of sensing layers 30 are connected to a hydroxyl group 50, respectively, for hydroxylating the surface thereof. According to the present embodiment, due to the shielding of the shielding layer 40, the hydroxyl group 50 is only disposed on the sensing area 32 of the sensing layer 30 to hydroxylate the surface of the sensing layer 30 in the sensing area 32. Then, a self-assembled monolayer film 52 including the aldehyde group is formed on the hydroxylated sensing area 32. Afterwards, according to the method for manufacturing a chip for quantitative detection of neutralizing antibody as described above, drip the protein solution on the sensing area 32 and apply an external electric field E at an external angle θ to the sensing layer 32 to deflect the protein molecules 2 in the protein solution and bind with the self-assembled monolayer film 52. According to the present embodiment, the external angle θ is the angle between the positive and negative electrodes of the external electric field E and the normal of the substrate 10.

According to the present embodiment, according to the present embodiment, the protein molecules 2 then bind with the target ligands to be detected. By limiting the locations of the hydroxyl group 50 and the protein molecules 2, the binding efficiency between the protein molecules and the target ligands to be detected can be enhanced and thus achieving the efficacy of quantitative detecting target ligands.

According to the present embodiment, according to the present embodiment, as shown in FIG. 12, when the external angle θ of the external electric field E is between −15° and 15°, the binding force of the protein molecules 2 is optimum. Thereby, according to the present embodiment, the external angle θ is between −15° and 15°. According to an embodiment, the external angle θ is preferably 0°.

According to the present embodiment, according to the present embodiment, a plurality of hydroxyl groups 50 and a plurality of protein molecules 2 are disposed in the sensing areas 32 of the sensing layer 30. Nonetheless, the present invention is not limited to the present embodiment.

According to the present embodiment, according to the present embodiment, the plurality of sensing layers 30 and the shielding layer 40 are the same as the above embodiment. The materials of the shielding layer 40 can avoid the hydroxyl group 50 from being disposed on the surface of the shielding layer 40, which will further cause the chip 1 for quantitative detection of neutralizing antibody to generate false signals.

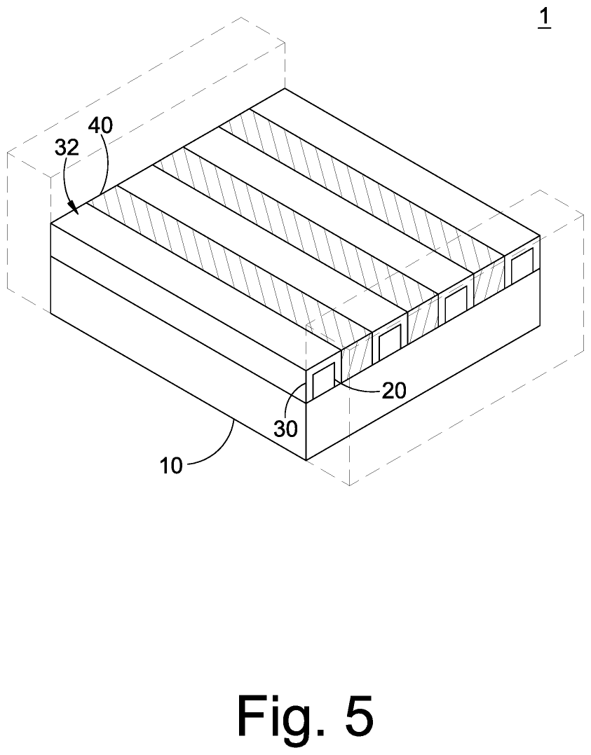

Please refer to FIG. 5, which shows a schematic diagram of the structure according to the third embodiment of the present invention. As shown in the figure, a detection chip 1 corresponding to the manufacturing method according to the third embodiment comprises a substrate 10, a plurality of circuit layers 20, a plurality of sensing layers 30, and a shielding layer 40. According to the present embodiment, the material of the substrate 10 includes, but not limited to, silicon.

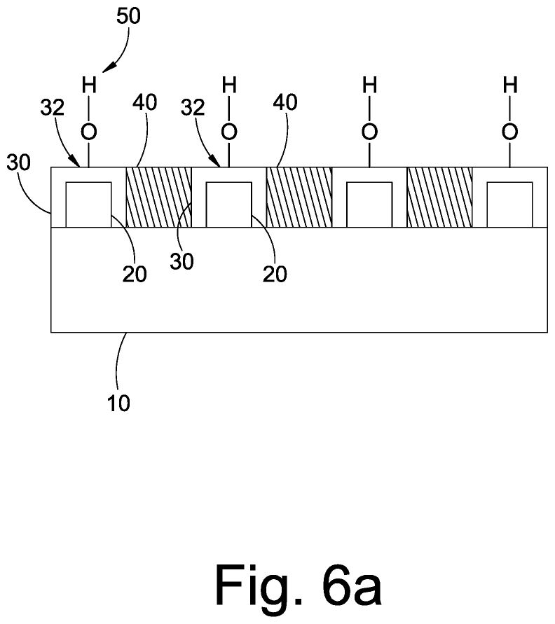

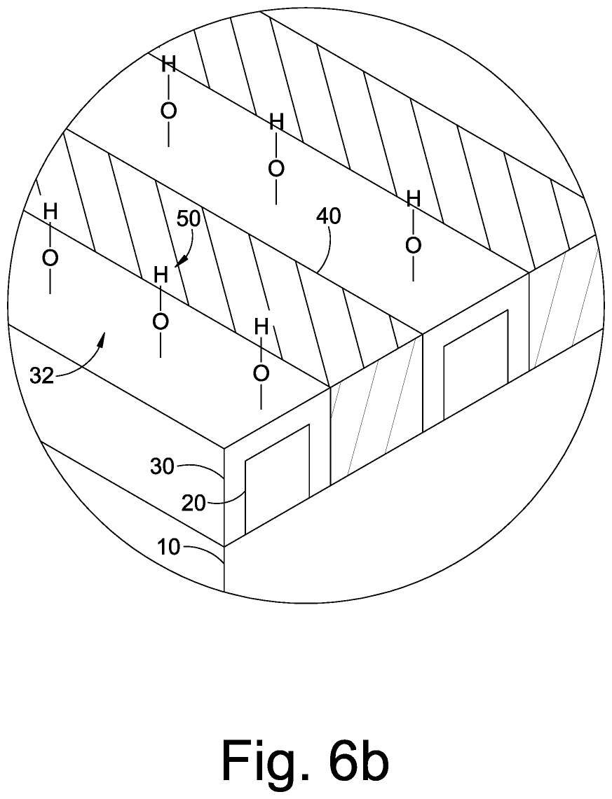

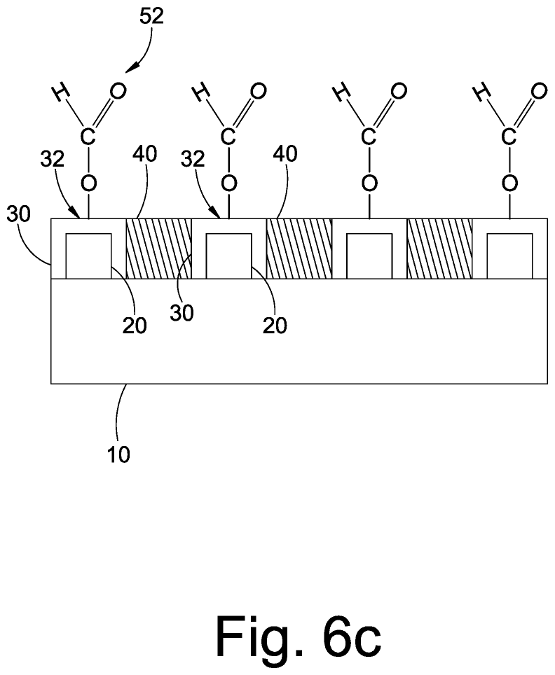

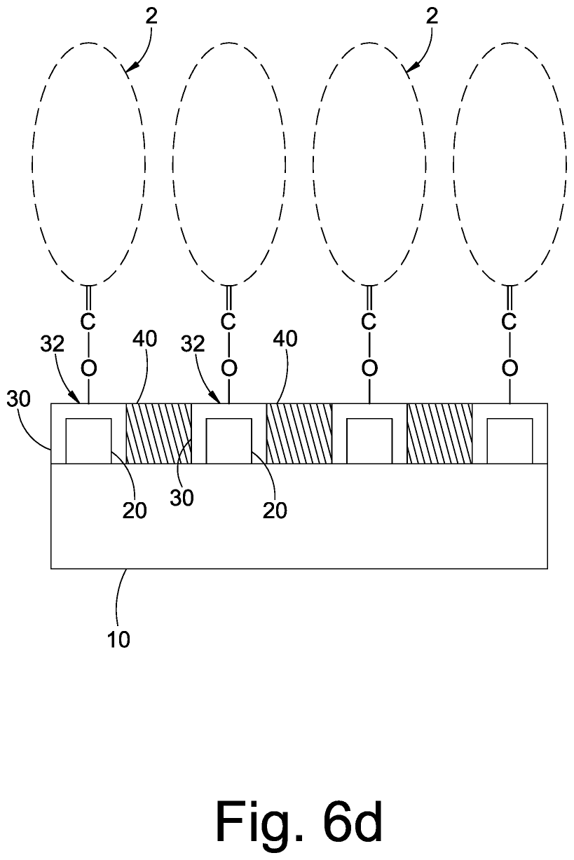

Please refer to FIG. 5 again and to FIGS. 6a to 6d, which show schematic diagrams of protein fixation according to the third embodiment of the present invention. As shown in the figures, according to the present embodiment, the plurality of circuit layers 20 are disposed on the substrate 10. The plurality of sensing layers 30 cover an outer side of the plurality of circuit layers 20, respectively. Namely, one of the plurality of sensing layers 30 covers the outer surface of one of the plurality of circuit layers 20, respectively. The shielding layer 40 is disposed on the substrate 10 and between the plurality of circuit layers 20. According to the present embodiment, the shielding layer 40 shields the surfaces between the plurality of sensing layers 30 and forms a sensing area 32 on the plurality of sensing layers 30, and thus giving a plurality of sensing areas 32.

The difference between the present embodiment and the first embodiment as described above is that, according to the present embodiment, the shielding layer 40 covers the surfaces between the plurality of sensing layers 30, respectively, for simplifying the manufacturing steps for the detection chip and further lowering the manufacturing cost for the chip 1 for quantitative detection of neutralizing antibody.

Please refer again to FIGS. 6a to 6d and FIG. 9. As shown in the figures, according to the present embodiment, the plurality of sensing layers 30 are connected to a hydroxyl group 50, respectively, for hydroxylating the surface thereof. According to the present embodiment, due to the shielding of the shielding layer 40, the hydroxyl group 50 is only disposed on the sensing area 32 of the sensing layer 30 to hydroxylate the surface of the sensing layer 30 in the sensing area 32. Then, a self-assembled monolayer film 52 including the aldehyde group is formed on the hydroxylated sensing area 32. Afterwards, according to the method for manufacturing a chip for quantitative detection of neutralizing antibody as described above, drip the protein solution on the sensing area 32 and apply an external electric field E at an external angle θ to the sensing layer 32 to deflect the protein molecules 2 in the protein solution and bind with the self-assembled monolayer film 52. According to the present embodiment, the external angle θ is the angle between the positive and negative electrodes of the external electric field E and the normal of the substrate 10.

According to the present embodiment, according to the present embodiment, the protein molecules 2 then bind with the target ligands to be detected. By limiting the locations of the hydroxyl group 50 and the protein molecules 2, the binding efficiency between the protein molecules and the target ligands to be detected can be enhanced and thus achieving the efficacy of quantitative detecting target ligands.

According to the present embodiment, according to the present embodiment, as shown in FIG. 12, when the external angle θ of the external electric field E is between −15° and 15°, the binding force of the protein molecules 2 is optimum. Thereby, according to the present embodiment, the external angle θ is between −15° and 15°. According to an embodiment, the external angle θ is preferably 0°.

According to the present embodiment, according to the present embodiment, a plurality of hydroxyl groups 50 and a plurality of protein molecules 2 are disposed in the sensing areas 32 of the sensing layer 30. Nonetheless, the present invention is not limited to the present embodiment.

According to the present embodiment, according to the present embodiment, the plurality of sensing layers 30 and the shielding layer 40 are the same as the above embodiment. The materials of the shielding layer 40 can avoid the hydroxyl group 50 from being disposed on the surface of the shielding layer 40, which will further cause the chip 1 for quantitative detection of neutralizing antibody to generate false signals.

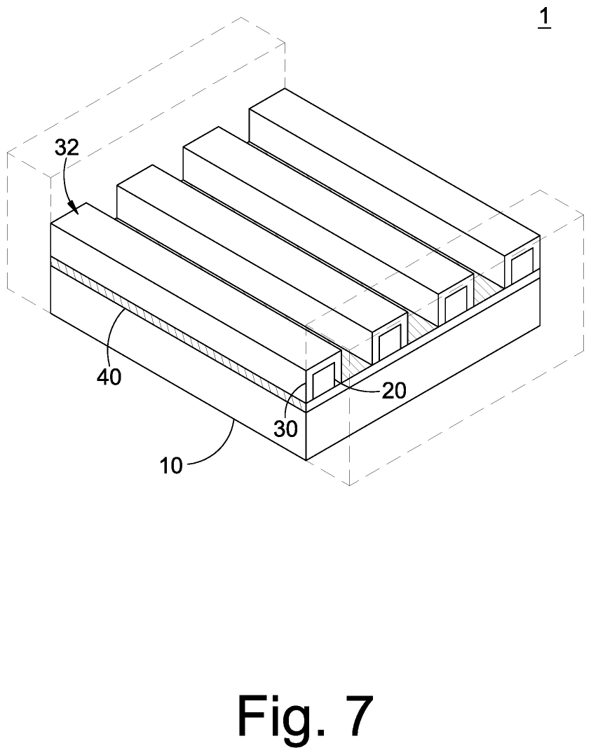

Please refer to FIG. 7, which shows a schematic diagram of the structure according to the fourth embodiment of the present invention. As shown in the figure, a detection chip 1 corresponding to the manufacturing method according to the fourth embodiment comprises a substrate 10, a shielding layer 40, a plurality of circuit layers 20, and a plurality of sensing layers 30. According to the present embodiment, the material of the substrate 10 includes, but not limited to, silicon.

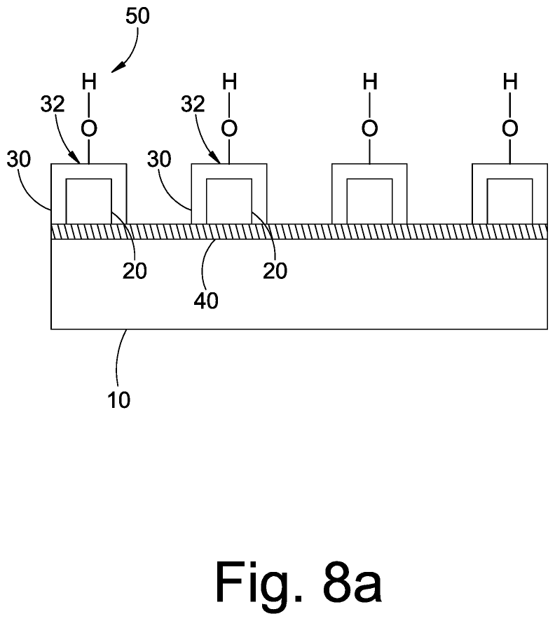

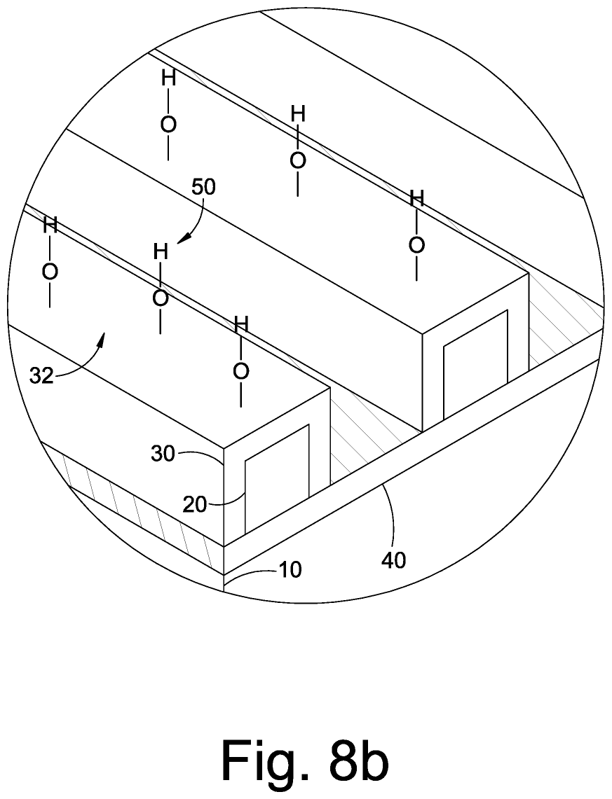

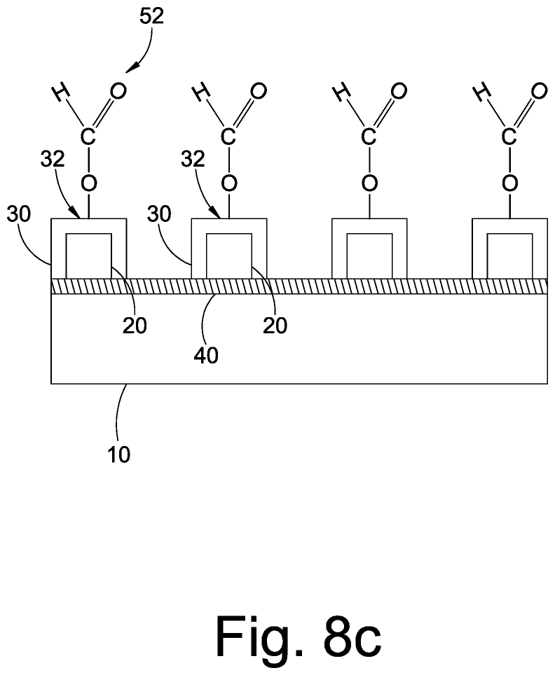

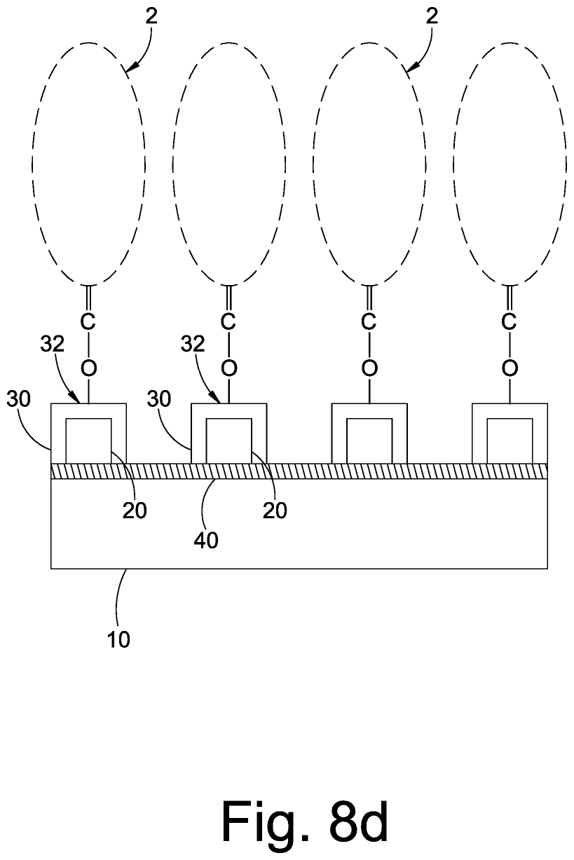

Please refer to FIG. 7 again and to FIGS. 8a to 8d, which show schematic diagrams of protein fixation according to the fourth embodiment of the present invention. As shown in the figures, according to the fourth embodiment, the shielding layer 40 is disposed on the substrate 10. The plurality of circuit layers 20 are then disposed on the shielding layer 40. The plurality of sensing layers 30 cover an outer side of the plurality of circuit layers 20, respectively. Namely, one of the plurality of sensing layers 30 covers the outer surface of one of the plurality of circuit layers 20, respectively. According to the present embodiment, the shielding layer 40 shields the substrate 10. The plurality of sensing layers 30 form a sensing area 32 on the outer side, and thus giving a plurality of sensing areas 32.

The difference between the present embodiment and the first embodiment as described above is that, according to the present embodiment, the shielding layer 40 is disposed between the substrate 10 and the plurality of circuit layers 20 for further simplifying the manufacturing steps for the detection chip and lowering the manufacturing cost for the chip 1 for quantitative detection of neutralizing antibody.

Please refer again to FIGS. 8a to 8d and FIG. 9. As shown in the figures, according to the present embodiment, the plurality of sensing layers 30 are connected to a hydroxyl group 50, respectively, for hydroxylating the surface thereof. According to the present embodiment, due to the shielding of the shielding layer 40, the hydroxyl group 50 is only disposed on the sensing area 32 of the sensing layer 30 to hydroxylate the surface of the sensing layer 30 in the sensing area 32. Then, a self-assembled monolayer film 52 including the aldehyde group is formed on the hydroxylated sensing area 32. Afterwards, according to the method for manufacturing a chip for quantitative detection of neutralizing antibody as described above, drip the protein solution on the sensing area 32 and apply an external electric field E at an external angle θ to the sensing layer 32 to deflect the protein molecules 2 in the protein solution and bind with the self-assembled monolayer film 52. According to the present embodiment, the external angle θ is the angle between the positive and negative electrodes of the external electric field E and the normal of the substrate 10.

According to the present embodiment, according to the present embodiment, the protein molecules 2 then bind with the target ligands to be detected. By limiting the locations of the hydroxyl group 50 and the protein molecules 2, the binding efficiency between the protein molecules and the target ligands to be detected can be enhanced and thus achieving the efficacy of quantitative detecting target ligands.

According to the present embodiment, according to the present embodiment, as shown in FIG. 12, when the external angle θ of the external electric field E is between −15° and 15°, the binding force of the protein molecules 2 is optimum. Thereby, according to the present embodiment, the external angle θ is between −15° and 15°. According to an embodiment, the external angle θ is preferably 0°.

According to the present embodiment, according to the present embodiment, a plurality of hydroxyl groups 50 and a plurality of protein molecules 2 are disposed in the sensing areas 32 of the sensing layer 30. Nonetheless, the present invention is not limited to the present embodiment.

According to the present embodiment, according to the present embodiment, the plurality of sensing layers 30 and the shielding layer 40 are the same as the above embodiment. These materials of the shielding layer 40 can avoid the hydroxyl group 50 from being disposed on the surface of the shielding layer 40, which will further cause the chip 1 for quantitative detection of neutralizing antibody to generate false signals.

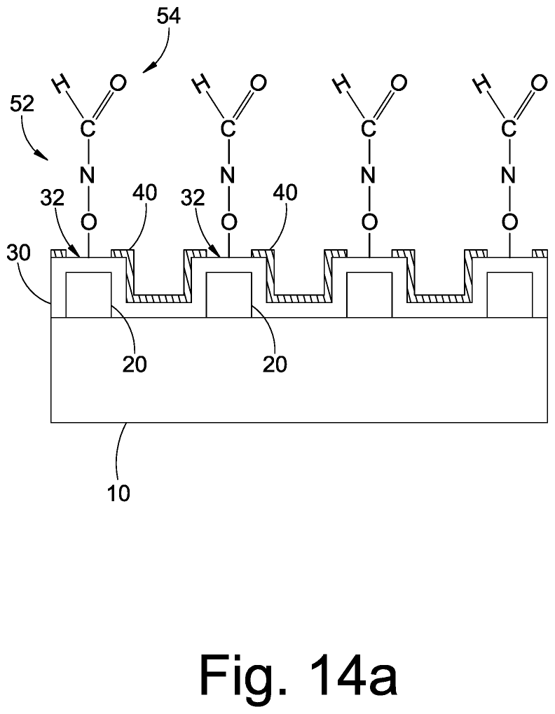

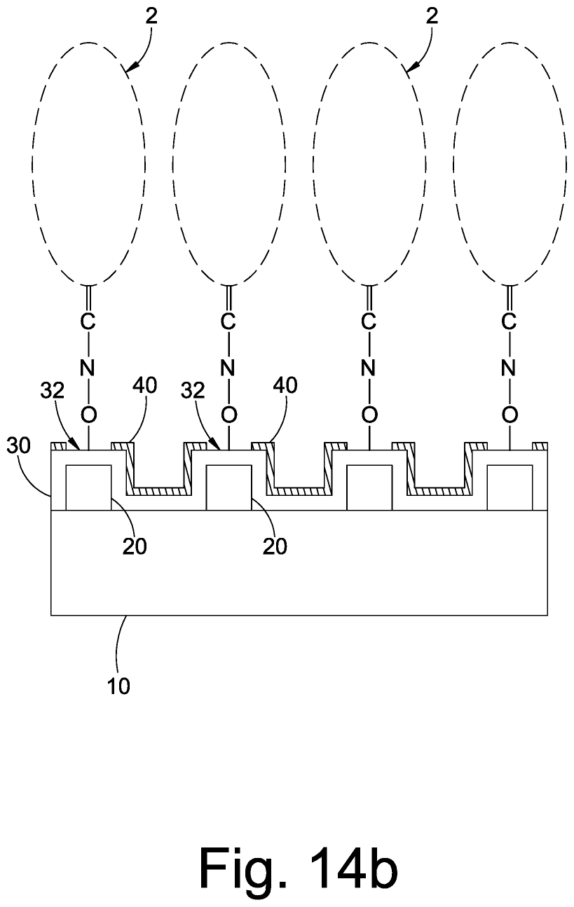

Please refer to FIGS. 14a to 14b, which show schematic diagrams of protein fixation according to another embodiment of the present invention. As shown in the figures, the present embodiment is based the first, second, third, and fourth embodiments as described above. Here, the first embodiment is taken as an example. After hydroxylating the surface of the plurality of sensing areas 32 of the detection chip 1 and forming the self-assembled monolayer film 52, a cross-linked molecular film 54 is further formed on the self-assembled monolayer film 52. Afterwards, according to the method for manufacturing a chip for quantitative detection of neutralizing antibody as described above, drip the protein solution on the sensing area 32 and apply the external electric field E at the external angle θ to the sensing layer 32 to deflect the protein molecules 2 in the protein solution and bind with the cross-linked molecular film 54.

To sum up, the present invention provides a chip for quantitative detection of neutralizing antibody and the manufacturing method thereof. A sensing layer is disposed on a circuit layer. Then a shielding layer shields a part of the sensing layer. The shield layer includes an opening corresponding to the circuit layer to expose a part of the sensing layer and forming a sensing area. The sensing area includes the hydroxyl group for further binding with protein molecules and sensing the target ligands to be detected. The shielding layer is used to limit the region on the sensing layer to bind the hydroxyl group, stabilize the signal generated by the sensing layer, and limit the locations of the hydroxyl group and protein molecules. In addition, by using the angle −15°˜15° of the external electric field with respect to the substrate, protein molecules are fixed to the chip for quantitative detection of neutralizing antibody (for example, the unknown antibody induced by the COVID-19 virus or vaccine), so that the chip for quantitative detection of neutralizing antibody can quantitatively detecting the concentration of the target ligands. The present invention solves the problem of random orientation of protein molecules on the detection chip in the process of fixing protein molecules to the detection chip according to the prior art. The random orientation lowers the binding efficiency between the protein molecules and the target ligands to be detected and deteriorates the detection efficiency of the detection chip for neutralizing antibody.

Accordingly, the present invention conforms to the legal requirements owing to its novelty, nonobviousness, and utility. However, the foregoing description is only embodiments of the present invention, not used to limit the scope and range of the present invention. Those equivalent changes or modifications made according to the shape, structure, feature, or spirit described in the claims of the present invention are included in the appended claims of the present invention.

1. A method for manufacturing a chip for quantitative detection of neutralizing antibody, applied to binding protein molecules in a protein solution, and said manufacturing method comprises steps of:
dripping said protein solution to a chip for quantitative detection of neutralizing antibody, which chip is used to bind said protein molecules to a sensing area of said chip for quantitative detection of neutralizing antibody; and
applying an external electric field to said chip for quantitative detection of neutralizing antibody at an external angle with respect to the normal of said chip for quantitative detection of neutralizing antibody and said external electric field deflecting said protein molecules corresponding to said external angle.

2. The method for manufacturing a chip for quantitative detection of neutralizing antibody of claim 1, and before said step of dripping said protein solution to a chip for quantitative detection of neutralizing antibody, which step is used to bind said protein molecules to a sensing area of said chip for quantitative detection of neutralizing antibody, further comprising steps of:
hydroxylating said sensing area of said chip for quantitative detection of neutralizing antibody; and
forming a self-assembled monolayer film including the aldehyde group for binding said protein molecules to said sensing area.

3. The method for manufacturing a chip for quantitative detection of neutralizing antibody of claim 1, and after hydroxylating the surfaces of said plurality of sensing areas and forming said self-assembled monolayer film, further forming a cross-linked molecular film on the self-assembled monolayer film.

4. The method for manufacturing a chip for quantitative detection of neutralizing antibody of claim 1, wherein the receptor binding domain of said protein molecules belongs to the superfamily and subfamily of CoV_Spike_S1_RBD.

5. The method for manufacturing a chip for quantitative detection of neutralizing antibody of claim 4, wherein said external angle with respect to the normal of said chip for quantitative detection of neutralizing antibody is between −15° and 15°.

6. A chip for quantitative detection of neutralizing antibody, comprising:
a substrate;
a plurality of circuit layers, disposed on said substrate;
a sensing layer, disposed on said substrate and on said plurality of circuit layers; and
a shielding layer, disposed on said sensing layer and including an opening corresponding to said plurality of circuit layers, and said opening forming a sensing area on said sensing layer.

7. The chip for quantitative detection of neutralizing antibody of claim 6, and after hydroxylating the surface of said sensing area and forming a self-assembled monolayer film including the aldehyde group, dripping said protein solution to said sensing area and applying an external electric field to said sensing layer at an external angle with respect to the normal of said substrate for deflecting said protein molecules in said protein solution and binding with said self-assembled monolayer film.

8. The chip for quantitative detection of neutralizing antibody of claim 6, wherein the material of said shielding layer is selected from the group consisting of silicon mononitride and silicon oxynitride.

9. The chip for quantitative detection of neutralizing antibody of claim 7, and after hydroxylating the surfaces of said plurality of sensing areas and forming said self-assembled monolayer film, further forming a cross-linked molecular film on the self-assembled monolayer film.

10. The chip for quantitative detection of neutralizing antibody of claim 7, wherein the receptor binding domain of said protein molecules belongs to the superfamily and subfamily of CoV_Spike_S1_RBD.

11. The chip for quantitative detection of neutralizing antibody of claim 10, wherein said external angle with respect to the normal of said chip for quantitative detection of neutralizing antibody is between −15° and 15°.

12. A chip for quantitative detection of neutralizing antibody, comprising:
a substrate;
a plurality of circuit layers, disposed on said substrate;
a plurality of sensing layers, disposed on said plurality of circuit layers, respectively; and
a shielding layer, disposed on said substrate, between said plurality of circuit layers, respectively, and on said plurality of sensing layers, including an opening corresponding to said plurality of circuit layers, respectively, and said opening forming a sensing area on said plurality of sensing layers, respectively.

13. The chip for quantitative detection of neutralizing antibody of claim 12, and after hydroxylating the surface of said plurality of sensing areas and forming a self-assembled monolayer film including the aldehyde group, dripping said protein solution to said plurality of sensing areas and applying an external electric field to said plurality of sensing areas at an external angle with respect to the normal of said substrate for deflecting said protein molecules in said protein solution and binding with said self-assembled monolayer film.

14. The chip for quantitative detection of neutralizing antibody of claim 12, wherein the material of said shielding layer is selected from the group consisting of silicon mononitride and silicon oxynitride.

15. The chip for quantitative detection of neutralizing antibody of claim 14, and after hydroxylating the surfaces of said plurality of sensing areas and forming said self-assembled monolayer film, further forming a cross-linked molecular film on the self-assembled monolayer film.

16. The chip for quantitative detection of neutralizing antibody of claim 13, wherein the receptor binding domain of said protein molecules belongs to the superfamily and subfamily of CoV_Spike_S1_RBD.

17. The chip for quantitative detection of neutralizing antibody of claim 16, wherein said external angle with respect to the normal of said chip for quantitative detection of neutralizing antibody is between −15° and 15°.

18. A chip for quantitative detection of neutralizing antibody, comprising:
a substrate;
a plurality of circuit layers, disposed on said substrate;
a plurality of sensing layers, covering the outer side of said plurality of circuit layers, respectively; and
a shielding layer, disposed on said substrate and between said plurality of circuit layers to form a plurality of sensing areas on said plurality of sensing layers.

19. The chip for quantitative detection of neutralizing antibody of claim 18, and after hydroxylating the surface of said plurality of sensing areas and forming a self-assembled monolayer film including the aldehyde group, dripping said protein solution to said plurality of sensing areas and applying an external electric field to said plurality of sensing areas at an external angle with respect to the normal of said substrate for deflecting said protein molecules in said protein solution and binding with said self-assembled monolayer film.

20. The chip for quantitative detection of neutralizing antibody of claim 19, and after hydroxylating the surfaces of said plurality of sensing areas and forming said self-assembled monolayer film, further forming a cross-linked molecular film on the self-assembled monolayer film.

21. The chip for quantitative detection of neutralizing antibody of claim 18, wherein the material of said shielding layer is selected from the group consisting of silicon mononitride and silicon oxynitride.

22. The chip for quantitative detection of neutralizing antibody of claim 19, wherein the receptor binding domain of said protein molecules belongs to the superfamily and subfamily of CoV_Spike_S1_RBD.

23. The chip for quantitative detection of neutralizing antibody of claim 22, wherein said external angle with respect to the normal of said chip for quantitative detection of neutralizing antibody is between −15° and 15°.

24. A chip for quantitative detection of neutralizing antibody, comprising:
a substrate;
a shielding layer, disposed on said substrate;
a plurality of circuit layers, disposed on said shielding layer; and
a plurality of sensing layers, covering the outer side of said plurality of circuit layers, respectively, to form a plurality of sensing areas on said plurality of sensing layers.

25. The chip for quantitative detection of neutralizing antibody of claim 24, and after hydroxylating the surface of said plurality of sensing areas and forming a self-assembled monolayer film including the aldehyde group, dripping said protein solution to said plurality of sensing areas and applying an external electric field to said plurality of sensing areas at an external angle with respect to the normal of said substrate for deflecting said protein molecules in said protein solution and binding with said self-assembled monolayer film.

26. The chip for quantitative detection of neutralizing antibody of claim 24, wherein the material of said shielding layer is selected from the group consisting of silicon mononitride and silicon oxynitride.

27. The chip for quantitative detection of neutralizing antibody of claim 25, and after hydroxylating the surfaces of said plurality of sensing areas and forming said self-assembled monolayer film, further forming a cross-linked molecular film on the self-assembled monolayer film.

28. The chip for quantitative detection of neutralizing antibody of claim 25, wherein the receptor binding domain of said protein molecules belongs to the superfamily and subfamily of CoV_Spike_S1_RBD.

29. The chip for quantitative detection of neutralizing antibody of claim 28, wherein said external angle with respect to the normal of said chip for quantitative detection of neutralizing antibody is between −15° and 15°.
